(19) United States
(12) Patent Application Publication
(10) Pub. No.: US 2016/0128962 A1
(43) Pub. Date: May 12, 2016

(54) LOW DOSE ORAL PHARMACEUTICAL COMPOSITION OF ISOTRETINOIN

(71) Applicant: Sun Pharmaceutical Industries Limited, Mumbai (IN)

(72) Inventors: Harish Kumar MADAN, Sonepat (IN); Rathinasabapathy VENKATESHWARAN, Madurai (IN); Sumit MADAN, New Delhi (IN); Ravi KOCHHAR, Gurgaon (IN)

(21) Appl. No.: 14/958,467

(22) Filed: Dec. 3, 2015

Related U.S. Application Data
(63) Continuation of application No. PCT/IB2015/054080, filed on May 29, 2015.

(30) Foreign Application Priority Data
Oct. 1, 2014 (IN) 2827/DEL/2014

(51) Int. Cl. A61K 31/202

(52) U.S. Cl. CPC A61K 31/202

(57) ABSTRACT

The present invention provides a low dose oral pharmaceutical composition of isotretinoin having reduced food effect. The present invention further relates to a process for preparing the oral pharmaceutical composition of the present invention.

### FIELD OF THE INVENTION

The present invention provides a low dose oral pharmaceutical composition of isotretinoin having reduced food effect. The present invention further relates to a process for preparing the oral pharmaceutical composition of the present invention.

### BACKGROUND OF THE INVENTION

Isotretinoin is a retinoid (also known as 13-cis retinoic acid). Owing to its low water solubility, the oral bioavailability of isotretinoin is low. PCT Publication No. WO 00/25772 discloses that the presently marketed formulation of isotretinoin, i.e., Accutane®, contains isotretinoin at a mean particle size of about 100 μm resulting in only 20% oral bioavailability. Therefore, this application discloses a formulation of isotretinoin having a reduced particle size, thereby enhancing the oral bioavailability.

U.S. Pat. Nos. 7,435,427 and 8,367,102 cover the marketed formulation of Absorica®. These patents disclose capsules comprising a semi-solid suspension of isotretinoin containing at least two lipidic excipients, one having an HLB value equal to or greater than 10 and the other being an oily vehicle. These patents are based on the use of the “Lidose technology” to provide a formulation of isotretinoin with enhanced bioavailability.

Isotretinoin has a very high teratogenic potential. This drug may be prescribed only by or under the supervision of a consultant dermatologist. Therefore, reduction of dose in case of such a teratogenic drug is highly beneficial. Further, isotretinoin is known to have a “food effect”, i.e., its absorption is dependent on the presence of food in the stomach. Therefore, there is a need to develop a composition of isotretinoin which has a lower dose and reduced food effect. The present inventors have developed an oral pharmaceutical composition of isotretinoin wherein said composition has enhanced bioavailability, lower dose and reduced food effect in comparison to the marketed formulations of isotretinoin, i.e., Roaccutane® and Absorica®. These advantages would lead to better patient compliance.

### SUMMARY OF THE INVENTION

The present invention provides a low dose oral pharmaceutical composition of isotretinoin having reduced food effect. The oral pharmaceutical composition of the present invention comprises isotretinoin and a pharmaceutically acceptable excipient. The present composition is in the form of a dispersion which is further filled into capsules. The present invention further provides a process for preparing the oral pharmaceutical composition of the present invention. It also provides a method of treating acne by administering the oral pharmaceutical composition of the present invention.

### DETAILED DESCRIPTION OF THE INVENTION

In one aspect, the present invention provides a low dose oral pharmaceutical composition comprising isotretinoin and a pharmaceutically acceptable excipient.

In another aspect, the present invention provides a low dose oral pharmaceutical composition comprising isotretinoin and a pharmaceutically acceptable excipient, wherein said composition, when administered orally, provides an equivalent efficacy at a lower dose of isotretinoin in comparison to the marketed Absorica® capsules, wherein said dose is at least 10% lower.

In another aspect, the present invention provides a low dose oral pharmaceutical composition comprising isotretinoin and a pharmaceutically acceptable excipient, wherein said composition, when administered orally, provides an equivalent efficacy at a lower dose of isotretinoin in comparison to the marketed Absorica® capsules, wherein said dose is at least 20% lower.

In another aspect, the present invention provides a low dose oral pharmaceutical composition comprising isotretinoin and a pharmaceutically acceptable excipient, wherein said composition exhibits reduced food effect as indicated by comparable Cₘₐₓ and AUC in fasting and fed states.

In an embodiment of the above aspect, the composition exhibits a mean Cₘₐₓ of about 451.38 ng/mL under fed condition and a mean Cₘₐₓ of about 454.92 ng/mL under fasting condition.

In another embodiment of the above aspect, the composition exhibits a mean AUC of about 6514.86 ng·h/mL under fed condition and a mean AUC of about 5566.90 ng·h/mL under fasting condition.

In another embodiment of the above aspect, the composition, when administered orally, has a mean fed/fasted ratio of AUC of about 1.17 and a mean fed/fasted ratio of Cₘₐₓ of about 0.99.

In another aspect, the present invention provides a low dose oral pharmaceutical composition comprising:

(a) isotretinoin; and

(b) an oily vehicle.

In one embodiment of the above aspect, said composition comprises isotretinoin in an amount of about 1 mg to 100 mg, 5 mg to 50 mg, 10 mg to 40 mg, 9 mg to 36 mg, or 8 mg to 32 mg.

In another embodiment of the above aspect, said composition comprises isotretinoin in an amount of about 16 mg.

In another embodiment of the above aspect, said composition comprises isotretinoin in an amount of about 32 mg.

In another embodiment of the above aspect, said composition comprises isotretinoin in an amount of about 8 mg, 16 mg, 20 mg, 24 mg, 28 mg, or 32 mg.

In another embodiment of the above aspect, said composition is in the form of a dispersion which is further filled into capsules.

In another embodiment of the above aspect, the oily vehicle includes, but is not limited to, groundnut oil, olive oil, soybean oil, kernel oil, almond oil, safflower oil, sunflower oil, palm oil, sesame oil, canola oil, corn oil, castor oil, coconut oil, cotton seed oil, grape seed oil, and mixtures thereof.

In another embodiment of the above aspect, the oily vehicle is present in an amount of about 1% w/w to about 99% w/w by the total weight of the composition; preferably in an amount of about 10% w/w to about 95% w/w by the total weight of the composition.

In another embodiment of the above aspect, the oily vehicle is present in an amount of about 71% w/w to about 95% w/w by the total weight of the composition.

In another embodiment of the above aspect, the ratio of isotretinoin to the oily vehicle ranges from about 1:99 to about 99:1.

In another embodiment of the above aspect, the composition further comprises a surfactant.

In another embodiment of the above aspect, the surfactant includes, but is not limited to, anionic, cationic, or non-ionic surfactants; sorbitan fatty acid esters; polysorbates prepared from lauric, palmitic, stearic, and oleic acids; mononylphenyl ethers of polyethylene glycols such as nanoxynols; polyoxyethylene monoesters such as polyoxyethylethylene monostearate, polyoxyethylene monolaurate, and polyoxyethylene monooleate; dioctyl sodium sulfosuccinate; sodium lauryl sulphate; lecithin; fatty acid esters of propylene glycol; fatty acid esters of glycerol; poloxamers; and mixtures thereof.

In another embodiment of the above aspect, the surfactant is present in an amount of about 0.05% w/w to about 10.0% w/w by the total weight of the composition.

In yet another embodiment of the above aspect, the composition further comprises other excipients like antioxidants, preservatives, and alkaline stabilizers.

In yet another embodiment of the above aspect, the composition is free of wax.

In yet another embodiment of the above aspect, the composition comprises isotretinoin wherein the particle size distribution of isotretinoin is such that the D₉₀ is less than 60 μm, less than 55 μm, less than 50 μm, less than 45 μm, less than 40 μm, less than 35 μm, less than 30 μm, less than 25 μm, less than 20 μm, less than 15 μm, or less than 10 μm.

In yet another embodiment of the above aspect, the composition comprises isotretinoin wherein the particle size distribution of isotretinoin is such that the D₉₀ is less than 30 μm.

In another embodiment of the above aspect, the composition comprises isotretinoin wherein the particle size distribution of isotretinoin is such that the D₅₀ is less than 40 μm, less than 35 μm, less than 30 μm, less than 25 μm, less than 20 μm, less than 15 μm, less than 10 μm, or less than 5 μm.

In yet another embodiment of the above aspect, the composition comprises isotretinoin wherein the particle size distribution of isotretinoin is such that the D₅₀ is less than 15 μm.

In another embodiment of the above aspect, the composition comprises isotretinoin wherein the particle size distribution of isotretinoin is such that the D₁₀ is less than 20 μm, less than 18 μm, less than 17 μm, less than 15 μm, less than 12 μm, less than 10 μm, less than 8 μm, less than 7 μm, less than 5 μm, or less than 2 μm.

In yet another embodiment of the above aspect, the composition comprises isotretinoin wherein the particle size distribution of isotretinoin is such that the D₁₀ is less than 7 μm.

In yet another embodiment of the above aspect, the composition comprises isotretinoin wherein the particle size distribution of isotretinoin is such that the D₉₀ is less than 60 μm and the D₅₀ is less than 40 μm.

In yet another embodiment of the above aspect, the composition comprises isotretinoin wherein the particle size distribution of isotretinoin is such that the D₉₀ is less than 60 μm, D₅₀ is less than 40 μm, and D₁₀ is less than 20 μm.

In yet another embodiment, said oral pharmaceutical composition is stable when stored at 40° C. and 75% relative humidity or at 25° C. and 60% relative humidity for a period of at least three months or to the extent necessary for the use of the composition.

In another aspect, there is provided a process for the preparation of a low dose oral pharmaceutical composition comprising isotretinoin and an oily vehicle wherein the process comprises:

(a) dispersing isotretinoin in an oily carrier;

(b) milling the dispersion to get the desired particle size;

(c) adding one or more excipients to the above dispersion;

(d) optionally adding an oily carrier to the dispersion of step (c); and

(e) filling the dispersion into capsules.

In one embodiment of the above aspect, the oily carrier used in step (a) is present in an amount which is at least 25% w/w of the total amount of the oily carrier.

In still another aspect, the present invention provides a method of treating acne, musculoskeletal and connective tissue inflammations, emphysema, ulcerating diseases, cervical tumors in HIV positive women, lung cancer in smokers, skin cancer, neuroblastoma, recurrent prostate cancer, leukemia, high-grade glioma, head and neck cancers, multiple myeloma, gram-negative folliculitis, recalcitrant rosacea, pyoderma faciale, psoriasis, cutaneous lupus erythematosus, acne fulminans, squamous cell carcinoma, or cutaneous photoaging by administering to the individual in need thereof, a low dose oral pharmaceutical composition of the present invention.

In one embodiment of the above aspect, the present invention provides a method of treating acne by administering to the individual in need thereof, a low dose oral pharmaceutical composition of the present invention.

The term “isotretinoin” refers to isotretinoin in its crystalline or amorphous form, as well as its esters, salts, or derivatives thereof.

The term “low dose,” as used herein, refers to the dose of isotretinoin wherein said dose is at least 10% lower than the presently approved dose.

The bioequivalence is established by comparing pharmacokinetic parameters, for example, AUC and Cₘₐₓ of the pharmaceutical composition of the present invention with Absorica® formulation in healthy human subjects in fed as well as fasting conditions.

The term “AUC” refers to the area under the time/plasma concentration curve after administration of the pharmaceutical composition. AUC_{0-infinity} denotes the area under the plasma concentration versus time curve from time 0 to infinity; AUC₀₋ₜ denotes the area under the plasma concentration versus time curve from time 0 to time t.

The term “Cₘₐₓ” refers to the maximum concentration of isotretinoin in the blood following administration of the pharmaceutical composition.

The term “tₘₐₓ” refers to the time in hours when Cₘₐₓ is achieved following administration of the pharmaceutical composition.

The term “food effect” as used herein means food-drug interactions which either decrease or increase the extent of drug absorption. It refers to a relative difference in AUC, Cₘₐₓ, and/or tₘₐₓ of a drug, when said drug or a formulation thereof is administered orally to a human, concomitantly with food or in a fed state as compared to the same values when the same formulation is administered in a fasted state or without food. Isotretinoin shows a food effect, i.e., its absorption is dependent on the presence of food in the stomach.

The term “D₁₀” refers to the particle size of isotretinoin where 10% (w/v) of the particles have a size less than the defined D₁₀ value; “D₅₀” refers to the particle size of isotretinoin where 50% (w/v) of the particles have a size less than the defined D₅₀ value; “D₉₀” refers to the particle size of isotretinoin where 90% (w/v) of the particles have a size less than the defined D₉₀ value.

“Defined D₁₀ value/D₅₀ value/D₉₀ value” refers to the values defined in the embodiments.

Examples of suitable antioxidants include, but are not limited to, butylated hydroxyl anisole, butylated hydroxyl toluene, tocopherol, ascorbyl palmitate, ascorbic acid, sodium metabisulfite, sodium sulfite, sodium thiosulfate, propyl gallate, and mixtures thereof. The antioxidant is present in an amount of about 0.002% w/w to about 2% w/w of the total composition.

Examples of alkaline stabilizers include, but are not limited to, sodium hydroxide, potassium hydroxide, sodium carbonate or bicarbonate, potassium carbonate or bicarbonate, lithium hydroxide, triethylamine, meglumine, methylamine, and mixtures thereof.

Examples of suitable preservatives include, but are not limited to, methyl paraben, ethyl paraben, propyl paraben, butyl paraben, benzoic acid, sodium benzoate, benzyl alcohol, sorbic acid, potassium sorbate, and mixtures thereof.

The term “stable,” as used herein, refers to chemical stability, wherein not more than 1.5% w/w of total related substances are formed on storage at accelerated conditions of stability at 40° C. and 75% relative humidity or at 25° C. and 60% relative humidity for a period of at least three months or to the extent necessary for use of the composition.

The size reduction of isotretinoin is achieved by wet milling the dispersion of isotretinoin in an oily vehicle using mechanical means such as a jet mill, ball mill, or media mills such as a sand mill, DYNO®-mill, or a bead mill. The grinding media in these mills can comprise spherical particles such as stainless steel beads or zirconium oxide balls.

The invention may be further illustrated by the following examples, which are for illustrative purposes only and should not be construed as limiting the scope of the invention in any way.

### EXAMPLES

### Example 1

| S. No. | Ingredients | Quantity (% w/w) |
|---|---|---|
| 1 | Isotretinoin | 6.67 |
| 2 | Butylated hydroxy anisole | 0.04 |
| 3 | Polysorbate 80 | 1.85 |
| 4 | Soybean oil | 91.44 |

### Procedure:

1. Butylated hydroxy anisole and polysorbate 80 were dissolved in soybean oil (39.36% w/v of the total composition) to form a clear solution.
2. Isotretinoin was added to the step 1 solution under stirring to obtain a uniform dispersion.
3. The dispersion of step 2 was milled to get the particle size of isotretinoin such that D₉₀ was about 20 μm.
4. The remaining quantity of soybean oil (52.08% w/v of the total composition) was added to the micronized dispersion of step 3 under stirring to obtain a uniform dispersion.
5. The dispersion of step 5 was filled into hard gelatin capsules.

### Dissolution Studies

The pharmaceutical composition of Example 1 (containing 16 mg of isotretinoin) was compared with the marketed formulation of isotretinoin (20 mg Absorica® capsules) for the release profile in an FDA recommended dissolution medium as given in the following tables:

Reference (R): Absorica® 20 mg capsules
Test (T): Isotretinoin 16 mg capsules (Example 1)

| | |
|---|---|
| Dissolution Media | pH 7.8 phosphate buffer with 0.5% w/v N,N-dimethyl |
| | dodecylamine N-oxide |
| Apparatus/RPM/Vol | USP Type I (20 mesh basket)/100/900 mL |

| | % of Drug Released Over Time (minutes) | | | | | | | | |
|---|---|---|---|---|---|---|---|---|---|
| Sample | 10 | 15 | 20 | 30 | 45 | 60 | 90 | 120 | 150 |
| Test | 34 | — | 58 | 73 | 93 | 99 | 100 | 101 | 100 |
| Reference | 0 | — | 2 | 6 | 24 | 37 | 58 | 76 | 83 |

### Pharmacokinetic Study Under Fed Conditions

The pharmaceutical composition of Example 1 (containing 16 mg of isotretinoin) was compared with the marketed formulation of isotretinoin (20 mg Absorica® capsules) under fed conditions on 15 healthy adult male subjects.

Values for various pharmacokinetic parameters, including observed Cₘₐₓ, AUC₀₋ₜ, and AUC_{0-inf} were calculated and are provided in Table 1 below.

Reference (R): Absorica® 20 mg capsules
Test (T): Isotretinoin 16 mg capsules (Example 1)

TABLE 1
Comparative pharmacokinetic data for test and reference in 15 healthy
adult human male subjects:

| | In Cₘₐₓ | In AUC₀₋ₜ | In AUC_{0-inf} |
|---|---|---|---|
| Ratio (T/R) | 111.07 | 90.12 | 91.59 |
| 90% CI | 91.54-134.76 | 84.30-96.35 | 86.32-97.19 |

Average tₘₐₓ values for both the test and reference are 4.7888 hours and 5.5111 hours, respectively, which indicate a comparable absorption pattern.
Under fed conditions, the test prototype shows a comparable extent of absorption to reference product with T/R ratios of 90.12% and 91.59% for AUC₀₋ₜ and AUC_{0-inf}, respectively. These values are within the regulatory acceptance criteria of 80% to 125%. However, for rate of absorption (Cₘₐₓ), the ratio is observed to be slightly on a higher side (111.07%) with 90% CI ranging between 91.54% and 134.76%.

### Pharmacokinetic Study Comparing the Formulation of Example 1 Under Fed and Fasting Conditions

The pharmaceutical composition of Example 1 (16 mg Test capsule) was compared in fed and fasting conditions on 15 healthy adult male subjects.

Values for various pharmacokinetic parameters, including observed Cₘₐₓ, AUC₀₋ₜ, and AUC_{0-inf} were calculated and are provided in Table 2 below.

Test (A): Isotretinoin 16 mg capsules (Example 1) under fasting conditions
Test (B): Isotretinoin 16 mg capsules (Example 1) under fed conditions

TABLE 2
Comparative pharmacokinetic data for test (B) vs test (A)
in 15 healthy adult human male subjects:

| | In Cₘₐₓ | In AUC₀₋ₜ | In AUC_{0-inf} |
|---|---|---|---|
| Ratio (B/A) | 99.22 | 116.34 | 117.02 |
| 90% CI | 81.78-120.38 | 108.82-124.37 | 110.29-124.17 |

Average tₘₐₓ for the test prototype under fasting condition (3.7667 hours) is ˜1.02 hours earlier than when administered under fed condition (4.7888 hours).
On comparing the test prototype under fasting and fed conditions, it is observed that B/A ratio for rate of absorption (Cₘₐₓ) is close to unity (99.22%). Even though B/A ratios are on higher side for the AUC values, (approx. 116% to 117%), the 90% CI for all three PK parameters (Cₘₐₓ, AUC₀₋ₜ, and AUC_{0-inf}) are within the 80% to 125% regulatory acceptance criteria.

### Conclusion:

The 16 mg test prototype has comparable bioavailability to the reference product (Absorica® 20 mg) under fed conditions. This provides positive support for up to 20% reduction in the test dose.
There is no indication that food will significantly impact the rate and extent of drug absorption from the test prototype. In fact, we observe that T/R ratios and 90% CI for the PK parameters are within the 80% to 125% regulatory acceptance criteria.

### Example 2

| S. No | Name of Ingredient | Quantity (% w/w) |
|---|---|---|
| 1. | Isotretinoin | 13.91 |
| 2. | Polysorbate 80 | 3.86 |
| 3. | Butylated hydroxy anisole | 0.08 |
| 4. | Soybean Oil | 82.15 |

### Procedure:

1. Butylated hydroxy anisole and polysorbate 80 were dissolved in the soybean oil to form a clear solution.
2. Isotretinoin was added to the step 1 solution under stirring to obtain a uniform dispersion.
3. The dispersion of step 2 was milled to get the particle size of isotretinoin such that D₉₀ was about 20 μm.
4. The dispersion of step 3 was filled into hard gelatin capsules.
5. The filled capsules of step 4 were sealed using a gelatin solution.

### Example 3

| S. No | Name of Ingredient | Quantity (% w/w) |
|---|---|---|
| 1. | Isotretinoin | 6.67 |
| 2. | Butylated Hydroxy Anisole | 0.04 |
| 3. | Soybean Oil | 93.29 |

### Procedure:

1. Butylated hydroxy anisole was dissolved in soybean oil (39.36% w/v of the total composition) to form a clear solution.
2. Isotretinoin was added to the step 1 solution under stirring to obtain a uniform dispersion.
3. The dispersion of step 2 was milled to get the particle size of isotretinoin such that D₉₀ was about 20 μm.
4. The remaining quantity of soybean oil (53.93% w/v of the total composition) was added to the micronized dispersion of step 3 under stirring to obtain a uniform dispersion.
5. The dispersion of step 4 was filled into hard gelatin capsules.
6. The filled capsules of step 5 were sealed using a gelatin solution.

1. (canceled)

2. A low dose oral pharmaceutical composition comprising isotretinoin and a pharmaceutically acceptable excipient, wherein said composition, when administered orally, provides equivalent efficacy at a lower dose of isotretinoin in comparison to the marketed Absorica®) capsules, wherein said dose is at least 10% lower.

3. The low dose oral pharmaceutical composition according to claim 2, wherein said composition, when administered orally, provides equivalent efficacy at a lower dose of isotretinoin in comparison to the marketed Absorica® capsules, wherein said dose is at least 20% lower.

4. The low dose oral pharmaceutical composition according to claim 2, wherein said composition exhibits a reduced food effect as indicated by comparable Cₘₐₓ, and AUC in fasting and fed state.

5. The low dose oral pharmaceutical composition according to claim 4, wherein said composition exhibits a mean Cₘₐₓ of about 451.38 ng/mL under fed condition and a mean Cₘₐₓ of about 454.92 ng/mL under fasting condition.

6. The low dose oral pharmaceutical composition according to claim 4, wherein said composition exhibits a mean AUC of about 6514.86 ng·h/mL under fed condition and a mean AUC of about 5566.90 ng·h/mL under fasting condition.

7. The low dose oral pharmaceutical composition according to claim 4, wherein the composition, when administered orally, has a mean fed/fasted ratio of AUC of about 1.17 and a mean fed/fasted ratio of Cₘₐₓ of about 0.99.

8. The low dose oral pharmaceutical composition according to claim 2, wherein said composition comprises:
(a) isotretinoin; and
(b) an oily vehicle.

9. The low dose oral pharmaceutical composition according to claim 8, wherein isotretinoin is present in an amount of about 1 mg to 100 mg, 5 mg to 50 mg, 10 mg to 40 mg, 9 mg to 36 mg, or 8 mg to 32 mg.

10. (canceled)

11. (canceled)

12. (canceled)

13. (canceled)

14. The low dose oral pharmaceutical composition according to claim 9, wherein the composition comprises isotretinoin in an amount of about 8 mg, 16 mg, 20 mg, 24 mg, 28 mg, or 32 mg.

15. The low dose oral pharmaceutical composition according to claim 8, wherein the composition is in the form of a dispersion which is further filled into capsules.

16. The low dose oral pharmaceutical composition according to claim 8, wherein the oily vehicle is selected from the group consisting of groundnut oil, olive oil, soybean oil, kernel oil, almond oil, safflower oil, sunflower oil, palm oil, sesame oil, canola oil, corn oil, castor oil, coconut oil, cotton seed oil, grape seed oil, and combinations thereof.

17. The low dose oral pharmaceutical composition according to claim 16, wherein the oily vehicle is present in an amount of about 1% w/w to about 99% w/w by the total weight of the composition.

18. The low dose oral pharmaceutical composition according to claim 17, wherein the oily vehicle is present in an amount of about 71% w/w to about 95% w/w by the total weight of the composition.

19. The low dose oral pharmaceutical composition according to claim 8, wherein the ratio of isotretinoin to the oily vehicle ranges from about 1:99 to about 99:1.

20. The low dose oral pharmaceutical composition according to claim 8, wherein said composition further comprises a surfactant.

21. The low dose oral pharmaceutical composition according to claim 20, wherein the surfactant is selected from the group consisting of anionic, cationic, or non-ionic surfactants; sorbitan fatty acid esters; polysorbates prepared from lauric, palmitic, stearic, and oleic acids; mononylphenyl ethers of polyethyleneglycols such as nanoxynols; polyoxyethylene monoesters such as polyoxyethylethylene monostearate, polyoxyethylene monolaurate, and polyoxyethylene monooleate; dioctyl sodium sulfosuccinate; sodium lauryl sulphate; lecithin; fatty acid esters of propylene glycol; fatty acid esters of glycerol; poloxamers; and mixtures thereof.

22. The low dose oral pharmaceutical composition according to claim 20, wherein the surfactant is present in an amount of about 0.05% w/v to about 10.0% w/v by the total weight of the composition.

23. The low dose oral pharmaceutical composition according to claim 8, wherein said composition further comprises an antioxidant, a preservative, an alkaline stabilizer, or combinations thereof.

24. The low dose oral pharmaceutical composition according to claim 8, wherein the composition is free of wax.

25. The low dose oral pharmaceutical composition according to claim 8, wherein the particle size distribution of isotretinoin is such that the D₉₀ is less than 60 μm, less than 55 μm, less than 50 μm, less than 45 μm, less than 40 μm, less than 35 μm, less than 30 μm, less than 25 μm, less than 20 μm, less than 15 μm, or less than 10 μm.

26. (canceled)

27. The low dose oral pharmaceutical composition according to claim 8, wherein the particle size distribution of isotretinoin is such that the D₅₀ is less than 40 μm, less than 35 μm, less than 30 μm, less than 25 μm, less than 20 μm, less than 15 μm, less than 10 μm, or less than 5 μm.

28. (canceled)

29. The low dose oral pharmaceutical composition according to claim 8, wherein the particle size distribution of isotretinoin is such that the D₁₀ is less than 20 μm, less than 18 μm, less than 17 μm, less than 15 μm, less than 12 μm, less than 10 μm, less than 8 μm, less than 7 μm, less than 5 μm, or less than 2 μm.

30. (canceled)

31. (canceled)

32. The low dose oral pharmaceutical composition according to claim 8, wherein the distribution of isotretinoin particle sizes is such that the 1)₉₀ is less than 60 μm, D₅₀ is less than 40 μm, and D₁₀ is less than 20 μm.

33. The low dose oral pharmaceutical composition according to claim 8, wherein said oral pharmaceutical composition is stable when stored at 40° C. and 75% relative humidity or at 25° C. and 60% relative humidity for a period of at least three months.

34. A process for preparing the low dose oral pharmaceutical composition according to claim 8, wherein said process comprises:
(a) dispersing isotretinoin in an oily carrier;
(b) milling the dispersion to get the desired particle size;
(c) adding one or more excipients to the above dispersion;
(d) optionally adding an oily carrier to the dispersion of step (c); and
(e) filling the dispersion into capsules.

35. The process according to claim 34, wherein the oily carrier used in the step (a) is present in an amount which is at least 25% w/w of the total amount of the oily carrier.

36. (canceled)

37. The low dose oral pharmaceutical composition according to claim 8, wherein said composition is used for the treatment of acne,
musculoskeletal and connective tissue inflammations, emphysema, ulcerating diseases, cervical tumors in HIV positive women, lung cancer in smokers, skin cancer, neuroblastoma, recurrent prostate cancer, leukemia, high-grade glioma, head and neck cancers, multiple myeloma, gram-negative folliculitis, recalcitrant rosacea, pyoderma faciale, psoriasis, cutaneous lupus erythematosus, acne fulminans, squamous cell carcinoma, or cutaneous photoaging.

38. The low dose oral pharmaceutical composition according to claim 37, wherein said composition is used for the treatment of acne.

39. (canceled)

40. (canceled)

41. The low dose oral pharmaceutical composition according to claim 2, wherein said composition releases more than 50% of isotretinoin in 20 minutes in a media with a pH of 7.8.
